(19) United States
(12) Patent Application Publication
(10) Pub. No.: US 2006/0285995 A1
(43) Pub. Date: Dec. 21, 2006

(54) Compositions and methods of use

(75) Inventors: Terry R. Hobbs, St. Paul, MN (US); David W. Kuhns, Minneapolis, MN (US)

Correspondence Address: 3M INNOVATIVE PROPERTIES COMPANY, PO BOX 33427, ST. PAUL, MN, 55133-3427, US

(73) Assignee: 3M Innovative Properties Company

(21) Appl. No.: 11/153,760

(22) Filed: Jun. 15, 2005

(51) Int. Cl. A61L 2/18

(52) U.S. Cl. 422/28

(57) ABSTRACT

A mycobactericidal composition is provided, comprising: a synergistic combination of a water miscible monohydric alcohol and benzoic acid; optionally, surfactant at a concentration less than about 1%; and water. A method for disinfecting a surface using the foregoing composition is also provided. The mycobactericidal compositions may be used for inactivating mycobacteria, bacteria, virus or fungi. In one embodiment, the composition of the invention is used for reconditioning a soiled endoscope.

The present invention relates to a composition that is useful in disinfecting surfaces, methods of making the composition and methods of using the composition for the disinfection of surfaces.

### BACKGROUND

A disinfecting composition, when applied to a surface or the like, will kill a wide spectrum of microorganisms such as bacteria, fungi and viruses. The term “high level disinfectant” (“HLD”) generally designates a class of disinfecting agents capable of killing 10⁶ mycobacteria and possessing the ability to kill bacterial endospores, the most difficult of all microorganisms to kill. A high level disinfectant can reduce spore populations and at the same time destroy less hardy pathogens such as mycobacteria, fungi, bacteria, and viruses. A “sterilant” is an agent capable of killing 10⁶ bacterial endospores.

In health care fields, medical devices such as bronchoscopes, endoscopes, laparoscopes find utility in medical procedures that expose the devices to significant amounts of biological soil. All of these instruments are typically used in medical procedures in which the instrument is inserted into the body either through a natural orifice or through a surgical opening. Internal channels extending through the scope may be configured to carry optical fibers, surgical instruments, or the like. Optical fibers affixed extending through the channel of the scope can be fixed to a small camera to facilitate the visual examination and treatment of areas within the body. In some configurations, power can be conveyed through a channel of the scope to power a small light fixture which can be conveyed to an area of interest within the body to facilitate the examination of organs, joints or body cavities. In fact, surgical instruments such as electrosurgery probes or forceps may be passed through the channels of a scope, and the channels may also be used to deliver fluids or gas, to provide suction or even to pass sampling catheters therethrough.

Virtually any portion of the human body is accessible to an endoscope, and typical surgical sites include the ears, throat, urinary tract, lungs, intestines and the abdominal cavity. Endoscopes used in colonoscopy procedures permit the direct examination of the inside of the colon and large intestines for the presence of polyps, ulcers and inflammation. Foreign bodies such as polyps or tumors may be surgically removed through the endoscope. As a consequence of their extensive use within the human body, endoscopes are exposed to biological soils that include blood, fecal matter, cellular matter from various tissue, and the like. Such biological soils can be sources of viruses, bacteria or other undesirable substances. In the United States and elsewhere, the endoscopes utilized by many medical or healthcare professionals are constructed to be re-usable, and re-usable endoscopes must be thoroughly cleaned and disinfected in a manner that ensures that the soiled surfaces are thoroughly disinfected prior to using the endoscopes in subsequent medical or surgical procedures.

Cleaning processes for reusable endoscopes are employed in which the soiled endoscope is initially cleaned during a manual cleaning step to remove as much soil as possible from all of the soiled surfaces of the instrument. Thereafter, a high level disinfection step is performed on the manually cleaned endoscope to render it ready for reuse. Typically, the manual cleaning step is performed by scrubbing the instrument with a brush or similar device in the presence of an enzymatic cleaning solution until soil can no longer be visually detected on the brush. Following manual cleaning, the endoscope is further disinfected by application of a high level disinfectant to the surfaces of the instrument. Substances used for of disinfecting the surfaces of medical instruments include peroxy compounds, hydrogen peroxide, chlorine compounds, aldehydes, and phenolics. These compounds and the compositions containing them have been used for disinfecting surfaces such as the lumen and other surfaces of any of a variety of medical devices. Mycobacteria are generally more difficult to kill in comparison to fungi, other bacteria, and viruses. Microorganisms from the Mycobacterium genus have been identified by the United States Food and Drug Agency (“FDA”) as the key organism to be used in establishing the disinfection time of a high level disinfectant. Tuberculosis, caused by Mycobacterium tuberculosis, is a key pathogenic organism of concern especially with the rise of antibiotic resistant strains. Approved non-pathogenic surrogates include Mycobacterium terrae and Mycobacterium bovis.

Products available for high level disinfection have often been slow in achieving a desired level of disinfection and may suffer from one or more other disadvantages. One example is glutaraldehyde at a 2% level in an aqueous solution. But, the disinfection times for glutaraldehyde products are often as long as 20 to 45 minutes. Although these disinfection times can be reduced with heating (e.g., to 35° C.), health issues have complicated the safety and efficacy picture for this compound. Likewise, peracetic acid and orthophthaldehyde have also been used in high level disinfection, but these compounds have generally provided undesirably lengthy disinfection times and/or have exhibited an undesirable material compatibility. Moreover, peracetic acid and orthophthaldehyde have exhibited concentration related health or safety issues. Hydrogen peroxide has also been used because of its broad germicidal properties with an ability to kill organisms through oxidative action. At lower concentrations (e.g., <6%), hydrogen peroxide is safe to handle and is considered environmentally friendly. But, hydrogen peroxide has also demonstrated a slow rate of disinfection, even when it has been used to eliminate common bacteria such as Staphyloccocus aureus (S. aureus). Although increased hydrogen peroxide concentrations can provide better kill rates, concentrated peroxide solutions are strong oxidizing agents, which can make them more hazardous to handle. Hydrogen peroxide concentrations of 8% or higher are classified by the United States Department of Transportation as strong oxidizers that require special shipping conditions.

There is a need for disinfectants capable of high level disinfection and exhibiting an improved rate of high level disinfection. Likewise, such a need exists for intermediate level disinfectants as well which provide a rapid reduction of 10⁶ bacteria such as S. Aureus or E. Coli. It is desirable to provide such a disinfectant in a safe and fast acting form capable of killing a broad range of microorganisms including mycobacteria, viruses, fungi, and bacteria while also having improved materials and skin compatibility.

### SUMMARY

In a first aspect, the invention provides a mycobactericidal composition comprising:
a synergistic combination of a water miscible monohydric alcohol and benzoic acid;
water; and
   optionally, surfactant at a concentration less than about 1% by weight.

In another aspect, the invention provides a method for disinfecting a surface, comprising the steps of:

Applying the foregoing composition to a surface.

In still another aspect, the invention provides a method for inactivating mycobacteria, bacteria, virus or fungi using the foregoing composition.

In still another aspect, the invention provides a method for reconditioning a soiled endoscope, comprising:
a first cleaning step to clean the surfaces of the endoscope;
leak testing the endoscope;
a second cleaning step to further clean the surfaces of the endoscope;
disinfecting the surfaces of the instrument by applying the above composition to the surfaces for a period of time;
rinsing the surfaces of the endoscope with water; and
drying the endoscope.

As used herein, the term “material compatibility” describes a property wherein the composition will not detrimentally effect or damage the surface material(s) to which the composition is applied. A determination of material compatibility may be made by immersing a material in a composition and thereafter analyzing the material by any of a variety of methods including a determination of weight gain or loss, changes in mechanical stiffness or compliance, by visual inspection, an observed change in color or shape, etc. . . . Material may characterized as compatible for a specified period of time (e.g., 5 minutes, 10 minutes, etc.) and incompatible if exposed for a longer period of time.

“Microorganism” or “microbe” or “microorganism” refers to bacteria, yeast, mold, fungi, protozoa, mycoplasma, as well as viruses (including lipid enveloped RNA and DNA viruses).

“Antiseptic” means a chemical agent that kills pathogenic and non-pathogenic microorganisms.

“Mucous membranes,” “mucosal membranes,” and “mucosal tissue” are used interchangeably and refer to the surfaces of the nasal (including anterior nares, nasoparangyl cavity, etc.), oral (e.g., mouth), outer ear, middle ear, vaginal cavities, and other similar tissues. Examples include mucosal membranes such as buccal, gingival, nasal, ocular, tracheal, bronchial, gastrointestinal, rectal, urethral, ureteral, vaginal, cervical, and uterine mucosal membranes.

“Subject” and “patient” includes humans, sheep, horses, cattle, pigs, dogs, cats, rats, mice, or other mammal.

As used herein, “a,” “an,” “the,” “at least one,” and “one or more” are used interchangeably. The term “and/or” means one or all of the listed elements (e.g., preventing and/or treating an affliction means preventing, treating, or both treating and preventing an affliction).

Those skilled in the art will further appreciate the various aspects of the invention upon consideration of the remainder of the disclosure. It is also contemplated that equivalents to the described components and to the composition of the invention are possible but are as yet unforeseen. Nonetheless, such equivalents are within the scope of the invention.

### DETAILED DESCRIPTION

The present invention provides disinfecting compositions. which are useful as low, intermediate level, and high level disinfectants for use on any of a variety of surfaces including living tissue such a mammalian skin and mucous membranes, for example. Additionally, the compositions of the invention may be used to as an industrial or a medical disinfectant on hard surfaces, textiles, and the surfaces of medical instruments (e.g., endoscopes).

In some embodiments, the invention provides compositions that are aqueous solutions comprising benzoic acid and a monohydric alcohol. When benzoic acid or monohydric alcohols are used individually in solution, they generally will exhibit no mycobactericidal activity at all or will exhibit such activity only to a very limited extent. Surprisingly, the combination of benzoic acid and monohydric alcohol according to the present invention act synergistically when used to kill mycobacteria. In some embodiments, compositions according to the invention exhibit rapid activity in that they are capable of killing 10⁶ mycobacteria within 2 minutes at 20° C. The compositions of the invention can be tailored for specific applications and, in some embodiments, may include additional components such as buffering salts, moisturizers, emollients, wetting agents, surfactants, corrosion inhibitors, solvents and sporicidal agents, for example.

The two principal components in the compositions of the invention, benzoic acid and monohydric alcohol, have been used extensively in the formulation of topical skin applications and are considered to be safe. Benzoic acid is widely used as a food preservative and has a long history of use in the treatment of fungal infections of the skin. Commercial formulations referred to “Whitfield's ointment” typically contain about 6 wt % benzoic acid for treating athlete's foot and ringworm. Likewise, water-soluble alcohols have been widely used at high concentrations on the skin as antiseptics.

In some embodiments, the compositions of the present invention will include benzoic acid at a concentration between about 0.01% and about 20% by weight of the solution. In some embodiments, the benzoic acid is present in amount between about 0.03% and about 5% by weight. In other embodiments, the benzoic acid content in the composition is less than 1% by weight.

In addition to benzoic acid, compositions according to the invention will include one or more monohydric alcohols in an amount that provides a synergistic effect when combined with benzoic acid. In other words, the inventive compositions of the invention comprise an amount of monohydric alcohol and benzoic acid that is more effective as a disinfecting composition than would be expected from the mere combination of the disinfecting properties of a separate monohydric alcohol solution and a benzoic acid solution. In some embodiments, the monohydric alcohol is present in an amount between about 1% and about 70% by weight. In some embodiments, the monohydric alcohol is present in amount between about 2% by weight to about 60% by weight. In still other embodiments, the monohydric alcohol is present in an amount between about 5% and about 30% by weight. In still other embodiments, the monohydric alcohol is present in amount between about 8% and about 20% by weight. Suitable alcohols include C₂-C₃ monohydric alcohols (e.g., ethanol, n-propanol and isopropanol) which are water miscible alcohols. In some embodiments where the composition comprises C₂-C₃ monohydric alcohols, the alcohol will consist of ethanol. In other embodiments, the alcohol will consist of n-propanol. In still other embodiments, the alcohol will consists of isopropanol. In still other embodiments, the alcohol will consists of a combination of two or more of the foregoing C₂-C₃ alcohols.

Those skilled in the art will appreciate that compositions of the invention can be applied to surfaces at ambient temperature or at elevated temperatures (e.g., higher than ambient temperature). In general, higher temperatures will result in higher kill rates for a given composition. Unless otherwise specified, references herein to the microbial kill achieved the compositions of the invention are being used at ambient temperature

Optional Components

The compositions of the invention may optionally include one or more surfactants capable of imparting desirable properties to the formulation such as improved wetting of surfaces, enhancement of cleaning properties, emulsification of skin conditioners, and possibly the enhancement of the antiviral properties of the compositions. When incorporated into an inventive composition that is intended to be used on skin, a criterion for the selection of an appropriate surfactant is that the surfactant can be characterized as ‘non-irritating’ when applied on human or mammalian skin at the concentration contemplated for that surfactant. However, counter-irritants may also be included in the compositions of the invention to counteract the possible irritating effects of a particular surfactant. When an optional surfactant is to be included in a composition according to the invention, the surfactant should generally be present at relatively low concentrations to avoid having the surfactant trap benzoic acid within the micelle formed by the surfactant. In some embodiments, the surfactant content is less than about 1 wt % and typically less than 0.25 wt %.

In some embodiments, surfactants suitable for use herein can include nonionic surfactants, anionic surfactants or combinations thereof. Suitable nonionic surfactants include, without limitation, alcohol ethoxylates, betaines, glucosides, fatty acid esters, amine oxides, sorbitan esters, and block copolymers of ethylene oxide and propylene oxide. Suitable block copolymers are commercially available under the trade designations “Pluronics” or “Lutrol” from BASF Corporation, Florham Park, N.J. Suitable anionic surfactants include alpha olefin sulfonates, alkyl benzene sulfonates, alkyl sulfates, fatty alcohol ethoxylate sulfonates, ester sulfosuccinates, diesters of sulfosuccinic esters, and salts of fatty acids. In some embodiments, the foregoing anionic surfactants are combined with counter-irritants which can include, without limitation, block copolymers of ethylene oxide and propylene oxide. In some embodiments, the anionic surfactant is sodium dioctyl sulfosuccinate.

Another optional component in the compositions of the invention is a sporicidal agent to assist in providing a high level disinfectant suitable for killing 10⁶ mycobacteria as well as destroying endospores. In embodiments of the invention, the compositions will have the ability to kill 10⁶ mycobacteria within predetermined exposure times. In some embodiments, the compositions have the ability to also kill endospores within a specified time at a certain temperature. In general, the exposure time required to kill endospores at a given temperature will be greater than the exposure time required for killing mycobacteria.

Suitable sporicidal agents are known to those skilled in the art. Such agents include, without limitation, those selected from hydrogen peroxide, peracids, peresters, chlorine iodine, povidone iodine, and aldehydes, as well as combinations of two or more of the foregoing. Typically, the concentration of sporicidal agents within the compositions of the invention will be within a range of from 0 to about 10% by weight. In some embodiments, the concentration of sporicidal agents will be within a range of from 0 to about 3% by weight. In embodiments where the sporicidal agent is hydrogen peroxide, the concentration of hydrogen peroxide will be within the range from about 2 to about 8% by weight.

In addition to the foregoing, optional components for the disinfecting solutions of the invention may also include buffering agents or salts, moisturizers, emollients, polymeric additives, wetting agents, and corrosion inhibitors. Moisturizers such as propylene glycol, glycerol, and lipids could be incorporated into a formulation to counteract any drying effect from the alcohol. Organic solvents and harsh detergents remove lipid layers found in the stratum corneum (the outermost layer of the skin) and decrease its barrier function resulting in dry skin. Moisturizers can immediately prevent excessive water loss from the skin, principally via occlusion. Occlusive moisturizing ingredients are oily substances that impair evaporation of skin moisture by forming a greasy film or layer that impedes water loss. Petrolatum is generally regarded as the most effective occlusive moisturizer. Other occlusive moisturizing ingredients include hydrocarbon agents such as mineral oil, paraffin, squalene, squalane, and fats such as cocoa butter, lanolin, stearic acid, and fatty alcohols. Cetyl alcohol is widely used in moisturizing lotions and creams. Other types of occlusives include wax esters, vegetable oils, fatty acid esters including beeswax, sterols, and silicones.

A second type of moisturizer is a humectant, a compound that attracts and holds water into the stratum coreum. These compounds are typically polar organic compounds that can hydrogen bond with water. Examples include propylene glycol, glycerin or glycerol, urea, sodium and potassium lactate, sorbitol, panthenol, and salts of pyyrolidone carboxylic acid.

A preferred polymeric additive for compositions of this invention is polyvinylpyrrolidone (PVP) and its copolymers. PVP can be used as multifunctional ingredient in the formulations of this invention. It forms water-soluble complexes with benzoic acid at higher concentrations and increases the solubility of benzoic acid in the formulation. Furthermore, it can also reduce the irritation caused by anionic surfactants such as sodium lauryl sulfate. It can also serve as a stabilizing agent, anti-soiling agent, and thickener.

The compositions of the invention may be provided in a concentrated form or in a more diluted or “ready to use” form. Concentrated versions of the compositions of the invention may be diluted at the point of use. Moreover, compositions of the invention may be further modified upon dilution by mixing a concentrated composition of the invention with another concentrate. For example, a composition comprising benzoic acid and alcohol may be provided in a concentrate and later mixed with a second concentrate containing peracetic acid. Additionally, the two concentrates could be volumetrically diluted with filtered water in a endoscope reprocessing unit resulting in a solution comprising alcohol, benzoic acid, and peracetic acid. The final diluted solution is useful as a disinfectant for medical devices.

Useful concentrates according to this invention may contain additional solvents, surfactants, hydrotropes, and sequestering agents. Surfactants may be present in some embodiments of the invention to prevent benzoic acid from precipitating when the concentrate is diluted with a large volume of water.

In embodiments of the invention, formulated as described herein, the compositions typically will have pH values less than about 7. In some embodiments, the pH will range from about 3.5 to about 6.5. The pH of a composition may be adjusted by adding an amine or a metal salt of benzoic acid or of another carboxylic acid. Suitable examples include but are not limited to sodium benzoate, potassium benzoate, triethanolamine benzoate, ammonium benzoate, sodium lactate, or the like. Similarly, inorganic salts may be added to the composition such as sodium phosphate or sodium hexametaphospate. The compositions of the invention are skin friendly and may be useful as a high level disinfectant for any of a variety of surfaces. Additionally, the compositions can be applied to the skin as a skin antiseptic for the hands or other areas of the body.

The compositions of the invention are generally useful as disinfectants of any of a variety of surfaces. The compositions are typically fast acting, safe and, because the monohydric alcohol inherently decreases the surface tension of the composition, can easily wet the surface to which the composition is applied. The compositions of the invention are useful as broad-spectrum disinfectants and antiseptics against tuberculosis, viruses, bacteria, and fungi, for example. In general, the compositions of the invention are effective against mycobacteria, perhaps the most difficult organisms to kill.

In using the compositions of the invention for the disinfection of surfaces, the composition is applied to the surface and is allowed to stand on the surface for a period of time. Contact times can vary within a wide range of time periods. In general, the contact times for the compositions of the invention can range from several seconds to about 30 minutes. Typically, the contact times will be about 10 minutes or less. The composition may then be removed from the surface by rinsing with water, for example. Alternatively, the composition may be allowed to evaporate from the surface either at ambient temperatures or by heating the surface. Compositions having higher alcohol levels will typically evaporate the fastest from a surface, whether heated or not.

In some embodiments, the inventive compositions may be formulated as hand or skin disinfectants and applied to the skin or to mucous membranes. For example, the compositions may be useful as hand sanitizers capable of disinfecting the skin and preventing the spread of pathogenic bacteria and viruses. The synergistic combination of benzoic acid and alcohol provides a disinfecting composition that is more effective against any of a variety of microbial contaminants including atypical mycobacterium. In some embodiments, the compositions of the invention may be used as pre-surgical preps or scrubs. In the foregoing embodiments of the invention, the composition may be applied to the skin with or without subsequent rinsing. In embodiments containing ingredients in addition to alcohol and benzoic acid such as emollients, for example, the composition may be applied to the skin without subsequent rinsing in order to obtain the most the beneficial effect of the emollient or other additional ingredient.

The composition of the present invention can also be used on the surfaces of medical instruments or devices including the surfaces of lumen. In particular, the composition of the present invention may be used in the reconditioning of a soiled endoscope. In this reconditioning method, the compositions of the invention are useful during the disinfection step of the cleaning process following use of the endoscope in a medical procedure.

In some embodiments, the foregoing method comprises:
a first cleaning step to clean the surfaces of the endoscope;
leak testing the endoscope;
a second cleaning step to further clean the surfaces of the endoscope;
disinfecting the surfaces of the instrument by applying the composition of claim 1 to the surfaces for a period of time;
rinsing the surfaces of the endoscope with water; and
drying the endoscope.
   In some embodiments, the disinfecting step is performed while the surfaces of the endoscope are at ambient temperature, the composition being applied to the surfaces for about 8 minutes or less. In other embodiments, the disinfecting step is performed while the surfaces of the endoscope are at an elevated temperature.

In further explanation of the foregoing process, a soiled endoscope is first subjected to a cleaning step in which the inner lumen and the outer surface of the endoscope is cleaned to remove gross debris remaining from the endoscope following a medical procedure. An enzymatic detergent is typically used in this cleaning step. Thereafter, the endoscope is leak tested to ensure that the inner channels of the endoscope are sufficiently protected from any seepage of fluids through the walls of the instrument. Following leak testing, both the outer surface of the instrument and the inner lumen are hand cleaned using a brush and an enzymatic detergent to remove remaining debris. Finally, the endoscope is subjected to a disinfection step in which the surfaces of the instrument are exposed to the composition of the invention for a specific period of time, either at ambient temperature or at an elevated temperature. If the exposure to the composition of the invention is at ambient or room temperature, an exposure time of about 8 minutes or less is typically sufficient to kill mycobacteria. It will be appreciated that an exposure to the compositions of the invention at an elevated temperature can shorten the exposure time needed to obtain an equivalent kill. Thereafter, the endoscope is rinsed with water and air dried. The reconditioned endoscope is then ready for use in another medical or surgical procedure.

### EXAMPLES

Additional embodiments of the invention are described in the following non-limiting Examples.

Test Procedures

### Quantitative Tuberculocidal Suspension

A 0.1 mL volume of Mycobacterium terrae (commercially available as ATCC 15755 from American Culture Collection of Rockville, Md.) grown in Middlebrook 7H9 Broth (commercially available from Difco Laboratories of Detroit, Mich.) with Middlebrook ADC Enrichment (available from Difco) was transferred to a 250 mL cell culture flask with a canted neck and a cap with a 0.2 μm filter containing 50 mL of Middlebrook 7H9 Broth supplemented with Middlebrook ADC Enrichment. The culture was incubated up to 2-4 weeks until the culture reached population around 10⁷ M. terrae cells/mL. On the same day that the examples were run, 6 mL of the culture was transferred into a tissue grinder and homogenized manually for 10 min. The uniformity of culture was checked using a microscope. The population of the working suspension was determined by diluting serially the bacterial solution in saline and plating onto the surface of Middlebrook 7H11 Agar supplemented with Middlebrook AODC Enrichment (available from Difco). The plates were incubated up to four weeks at 37° C. and CFUs were counted.

A small Erlenmeyer flask containing a magnetic stirring bar was filled with 9 mL of the HLD Example composition. The flask was placed on the magnetic stirrer and the solution was mixed for 10 minutes in a controlled temperature (approximately 20° C.) water bath, to assure uniformity of the solution. A 1.0 mL of working suspension containing 5% bovine calf serum (commercially available from Hyclone of Logan, Utah) was added to the HLD Example composition while stirring.

At the start of each exposure time, 1 mL of cell working suspension was added to the mixing compositions with soil. Typical exposure times consisted of various multiple time points as shown in the results for each Example. Various other time points were also evaluated. At the end of each exposure time, 1 mL of suspension was transferred to a test tube containing 9 mL DE broth as a neutralizer with 0.01 mL catalase. DE was Dey Engle broth purchased from Difco Laboratories of Detroit, Mich. After vortexing, the neutralized 10⁻¹ solution suspension was further diluted to 10⁻²-10⁻⁷ by transferring 1 mL into 9 mL DE dilution blanks. From each dilution, 0.1 mL volume was plated into TSA plate spread with the L-rod. In some cases the suspension was filtered trough a Millipore filter which was previously wetted with approximately 10 mL of saline. After the filtration of the neutralized bacterial suspension, the filter was rinsed with 50 mL of saline. The filter with bacteria was aseptically transferred onto Middlebrook 7H 11 agar plates supplemented with Enrichment AODC nutrients. The plates were incubated in a plastic bag to prevent drying at 35° C. for 2 weeks and CFUs were counted.

Mycobactericidal activity was reported as a log₁₀ reduction, which was determined by calculating the difference between the log₁₀ of the initial inoculum count and the log₁₀ of the inoculum count after exposure to the compositions or components of the composition for specified intervals of time. The calculations were described in the Microbial Kill Rate Assay.

Controls

A Static Control was used to establish the effectiveness of the neutralizer. A volume of 0.9 mL of the HLD was added to 9.0 mL DE neutralizer with catalase (available from Difco). Then 0.1 mL of the inoculum was be added to this solution and was treated identically to the test procedure. This procedure was repeated using sterile saline (saline blank control) in place of the neutralizer and test substance and the data was compared to the static control. The acceptance criteria for this study control require that the static control and corresponding population control results to be within 1.0 log.

A Toxicity Control was used to demonstrate the neutralizer's lack of toxic effect on the test organisms at the concentrations employed in this method. A volume of 0.9 mL of the diluent (saline) was added to 9.0 mL neutralizer and mixed. A volume of 0.1 mL of the inoculum was be added to this solution and was treated identically to the test procedure. The toxicity control will be processed as the HLD. The acceptance criterion for this study control requires that the toxicity neutralization control and corresponding population control results to be within 1.0 log.

A Neutralizer System Control was used to demonstrate the effectiveness of the neutralizer in conjunction with the washing procedure in neutralizing the test substance. A volume of 0.9 mL of the HLD was added to 9.0 mL neutralizer and mixed. A volume of 0.1 mL of sterile growth medium (7H9 broth) was added to this solution and will be treated identically to the test procedure. The solution was filtered and washed as the 10E-1 dilution. The filter will be inoculated with approximately 100 CFU, evacuated, and plated. The acceptance criterion for this study control requires the filtration neutralization control and corresponding population control results to be within 1.0 log.

Components used in the various Examples are listed in Table 1. Unless otherwise indicated, the components used were of food or pharmaceutical grade.

TABLE 1
Components

| | | | Commercial |
|---|---|---|---|
| Component | Trade Designation | Function/identity | Source/Address |
| Adipic acid | — | Aliphatic acid | Sigma-Aldrich |
| | | | Chemical Co./St. |
| | | | Louis, MO |
| Benzoic acid | — | Aromatic acid | Brenntag Great Lakes |
| | | | Chemical Co. St. |
| | | | Paul, MN |
| Benzotriazole | COBRATEC 35G | Corrosion inhibitor - | PMC Specialties |
| | | (35% in propylene | Inc./Cincinnati, OH |
| | | glycol) | |
| Benzotriazole | COBRATEC 99P | Corrosion inhibitor | PMC Specialties |
| | | | Inc./Cincinnati, OH |
| Ceteareth 20 | Brij 58 | Non-ionic surfactant | Uniqema/New |
| | | | Castlem DE |
| Decanol | — | Solvent | Proctor and Gamble/ |
| | | | Cincinnati, OH |
| Disodium EDTA | — | Chelating agent | Sigma-Aldrich |
| | | | Chemical Co./St. |
| | | | Louis, MO |
| Distilled water | water | Base/carrier | Premium Waters |
| | | | Inc./Minneapolis, |
| | | | MN |
| Ethanol | — | Solvent | EMD Chemicals Inc./ |
| | | | Gibbstown, NJ |
| Glycerin, USP | — | Moisturizer | Proctor and Gamble/ |
| | | | Cincinnati, OH |
| Hydrogen peroxide | SUPER D Stabilized | Peroxide source, | FMC Corp./ |
| | Hydrogen Peroxide | oxidizing agent | Philadelphia, PA |
| | (35% solution) | | |
| Isopropanol (2-propanol) | IPA | Solvent | EMD Chemicals Inc./ |
| | | | Gibbstown, NJ |
| n-propanol (1-propanol) | — | Solvent | EMD Chemicals Inc./ |
| | | | Gibbstown, NJ |
| Lactic acid | — | Aliphatic acid | Sigma-Aldrich |
| | | | Chemical Co., St. |
| | | | Louis, MO |
| Malic acid | — | Aliphatic acid | Sigma-Aldrich |
| | | | Chemical Co./St. |
| | | | Louis, MO |
| p-hydroxy benzoic acid | — | Aromatic acid | Sigma-Aldrich |
| | | | Chemical Co./St. |
| | | | Louis, MO |
| Phosphoric acid | — | Acidulant (85%) | J T Baker Co./ |
| | | | Phillipsburg, NJ |
| Polydimethylsiloxane | Antifoam C | Antifoaming agent - | Dow Corning/ |
| | | (30%) food grade | Midland, Michigan |
| poloxamer | Lutrol F68 | Nonionic surfactant | BASF Corp./ |
| | | | Florham Park, NJ |
| Polyvinyl pyrrolidone | PVP K90 | Solubility Enhancer/ | ISP Technologies Inc./ |
| | | crystallization | Wayne, NJ |
| | | inhibitor | |
| Propylene glycol | PG | USP grade Solvent, | Brenntag Great Lakes |
| | | wetting agent | Chemical Co./St. |
| | | | Paul, MN |
| Sodium benzoate USP | — | Salt of benzoic | Brenntag Great Lakes/ |
| | | acid (99%) | St. Paul, MN |
| Sodium dioctyl | AEROSOL OT | Anionic surfactant | Cytec Industries/ |
| sulfosuccinate | | (100%) | West Paterson, NJ |
| Sodium dodecyl | Biosoft D-40 | Anionic surfactant | Stepan Co./ |
| benzenesulfonate | | (40%) | Northfield, IL |
| Sodium hydroxide | NaOH | pH adjustment | Mallinkrodt/Paris, |
| | | | KY |
| Sodium lauryl sulfate | WA-Extra | Anionic surfactant | Stepan Co./ |
| | | | Northfield, IL |
| Tolyltriazole | COBRATEC TT100 | Corrosion inhibitor | PMC Specialties |
| | | | Inc./Cincinnati, OH |

### Comparative Examples C1-C2 and Examples 1-4

Examples 1-4 and Comparative Examples C1-C2 were prepared using the components listed in Table 1 and evaluated according to the Quantitative Tuberculocidal Suspension test procedure described above. The results are shown in Table 2.

TABLE 2

| | Example Numbers | | | | | |
|---|---|---|---|---|---|---|
| | C1 | C2 | 1 | 2 | 3 | 4 |
| Component | gram amounts of components | | | | | |
| Benzoic acid | 0.82 | — | 1.03 | 0.51 | 0.78 | 0.36 |
| Sodium benzoate | 0.76 | — | 0.49 | 0.27 | 0.83 | 0.64 |
| 35% Hydrogen Peroxide | 14.38 | 14.34 | — | — | — | — |
| Propylene glycol | 25.14 | 15.20 | 16.27 | — | 18.99 | — |
| AEROSOL OT | 0.74 | 0.46 | 0.46 | — | — | — |
| IPA | — | 10.00 | 9.67 | — | 9.53 | — |
| n-propanol | — | — | — | 15.55 | — | 15.00 |
| Glycerin USP | — | — | — | 4.03 | — | |
| Certeareth 20 (Brij 58) | — | — | — | 0.31 | — | — |
| Lutrol F68 | — | — | — | — | — | 0.10 |
| Water (distilled) | 58.12 | 60.00 | 72.24 | 78.55 | 69.93 | 84.53 |
| Total | 99.96 | 100.0 | 100.16 | 99.22 | 100.06 | 100.63 |
| pH | 4.3 | — | 4.3 | — | — | 4.7 |

Antimicrobial efficacy results for log reduction of mycobacterium terrae (ATCC15755):

| | | | | | | |
|---|---|---|---|---|---|---|
| 1 minute | — | — | 2.0 | — | — | — |
| 1.5 minutes | — | — | — | >6.3** | — | — |
| 2 minutes | — | — | 4.5 | — | — | 6.2 |
| 3 minutes | <3.1 | <0.5 | >6.1* | — | 3.6 | >6.5* |
| 5 minutes | 3.4 | — | — | — | — | — |

*Complete Kill.
**Example 2 had complete kill at 1.5 minutes and was tested at 35° C.

### Comparative Examples C3-C5 and Examples 5-7

Examples 5-7 and Comparative Examples C3-C5 were prepared to demonstrate the synergist interaction between a low level of alcohol and a low level of benzoic acid. All formulas with benzoic acid contain sodium benzoate as a buffering agent such that the pH is above 3.5. Propylene glycol is used as an additional solvent in Comparative Examples C4-C5 and Examples 5-6. Each Example was prepared by adding the solvent(s) to a 120 mL glass jar with a magnetic stir bar. The benzoic acid and sodium benzoate were added and stirred for 1 hour. The formulations were then diluted with deionized water. Example 6 has stabilized hydrogen peroxide as a second antimicrobial added as the last ingredient.

These Examples were evaluated in the mycobactericidal kill rate assay (suspension test) at 23° C. using three time points. The log reduction of each Examples is shown Table 3.

TABLE 3

| | Example Numbers | | | | | |
|---|---|---|---|---|---|---|
| | C3 | C4 | C5 | 5 | 6 | 7 |
| Component | gram amounts of components | | | | | |
| Benzoic acid | — | 0.75 | — | 0.75 | 0.75 | 0.75 |
| Sodium benzoate | — | 0.75 | — | 0.75 | 0.75 | 0.75 |
| 35% Hydrogen Peroxide | — | — | — | — | — | 10.00 |
| Propylene glycol | — | 25.00 | 25.00 | — | 25.00 | 25.00 |
| IPA (2-propanol) | 9.50 | — | 9.50 | 9.50 | 9.50 | 9.50 |
| Water | 90.50 | 73.50 | 65.50 | 89.00 | 64.00 | 54.00 |
| Total | 100.0 | 100.0 | 100.0 | 100.0 | 100.0 | 100.0 |
| pH | — | >3.5 | — | >3.5 | >3.5 | >3.5 |

Antimicrobial efficacy results for log reduction of mycobacterium terrae (ATCC15755):

| | | | | | | |
|---|---|---|---|---|---|---|
| 1 minute | 0.0 | 0.1 | 0.0 | 0.0 | 0.7 | 0.6 |
| 3 minutes | 0.0 | 0.6 | 0.0 | 5.0* | 3.2 | 3.8 |
| 5 minutes | — | 1.3 | — | >6.3** | 6.3 | 5.7 |

*Time point is 3.5 minutes.
**Complete kill.

### Comparative Examples C6-C8 and Examples 8-9

Examples 8-9 and Comparative Examples C6-C8 were prepared. Hand antiseptic Examples 8 and 9 were made with 7% propylene glycol USP and 3% glycerin USP as moisturizing agents. Comparative Examples C6 and C7 do not contain benzoic acid. The formulations were again tested in a mycobactericidal kill rate assay at 15, 30, and 45 seconds. The results are summarized in the Table 4.

TABLE 4

| | Example Numbers | | | | |
|---|---|---|---|---|---|
| | C6 | C7 | C8 | 8 | 9 |
| Component | gram amounts of components | | | | |
| Benzoic acid | — | — | 0.46 | 0.46 | 0.46 |
| Ethanol | 50.0 | 60.0 | — | 50.3 | 60.0 |
| Propylene | 7.00 | 7.00 | 7.02 | 7.03 | 6.99 |
| glycol, USP | | | | | |
| Glycerin, USP | 3.00 | 3.00 | 3.03 | 3.00 | 3.08 |
| Water | 40.00 | 30.00 | 90.52 | 39.66 | 29.77 |
| Total | 100.00 | 100.00 | 101.03 | 100.45 | 100.30 |

Antimicrobial efficacy results for log
reduction of mycobacterium terrae (ATCC15755):

| | | | | | |
|---|---|---|---|---|---|
| 15 seconds | 0.8 | 3.7* | 0.1 | 1.2 | 4.2 |
| 30 seconds | 2.8 | >6.7** | 0.0 | 5.3 | >6.7** |
| 45 seconds | 5.0 | >6.7** | 0.0 | >6.7** | >6.7** |

*Measured at 17 seconds.
**Complete kill.

### Examples 10-11 and Comparative Examples C9-C12

Examples 10-11 and Comparative Examples C9-C12, shown in Table 5 below, were prepared by combining the acid and alcohol; followed by the addition of water and other ingredients (PVP K-90). Table 5 shows the log reduction of Mycobacterium terrae for each Example tested in a suspension kill rate assay using a 5 minute exposure time at 20° C. Comparative Examples C9-C12 show no significant kill. Example C10 contains p-hydroxybenzoic acid, a structural analog of benzoic acid. Examples 10 and 11, containing benzoic acid in combination with n-propanol, show >5 log reduction indicating significant activity.

TABLE 5

| | Example Numbers | | | | | |
|---|---|---|---|---|---|---|
| | C9 | C10 | C11 | C12 | 10 | 11 |
| Component | gram amounts of components | | | | | |
| Acid type | malic | p-hydroxy | adipic | lactic | benzoic | benzoic |
| | | benzoic | | | | |
| Acid Amount in grams | 0.5 | 0.5 | 0.5 | 0.5 | 0.5 | 0.5 |
| n-propanol | 8.0 | 8.0 | 8.0 | 8.0 | 8.0 | 10.0 |
| Water | 91.5 | 91.5 | 91.5 | 91.5 | 91.5 | 90.5 |
| PVP K-90 | — | — | — | — | — | 0.8 |
| Total | 100 | 100 | 100 | 100 | 100 | 101.8 |

Antimicrobial efficacy results for log reduction of mycobacterium terrae (ATCC15755):

| | | | | | | |
|---|---|---|---|---|---|---|
| 5 minutes | 0.1 | 0.3 | 0.2 | 0.2 | 5.8 | 6.5 |

Various embodiments of the invention have been described as foreseen by the inventor for which an enabling description was available. It should be appreciated that insubstantial modifications of the invention, not presently foreseeable by those of reasonable skill in the art, may nonetheless represent equivalents thereto.

1. A mycobactericidal composition comprising:
a synergistic combination of a water miscible monohydric alcohol and benzoic acid;
water; and
optionally, surfactant at a concentration less than about 1% by weight.

2. A mycobactericidal composition as defined in claim 1 wherein the monohydric alcohol is selected from the group consisting of ethanol, n-propanol, 2-propanol and combinations of two or more of the foregoing.

3. A mycobactericidal composition as defined in claim 2 wherein the monohydric alcohol is present in an amount between about 1% and about 70% by weight.

4. A mycobactericidal composition as defined in claim 2 wherein the monohydric alcohol is present in an amount between about 2% and about 60% by weight.

5. A mycobactericidal composition as defined in claim 2 wherein the monohydric alcohol is present in an amount between about 5% and about 30% by weight.

6. A mycobactericidal composition as defined in claim 2 wherein the monohydric alcohol is present in an amount between about 8% and about 20% by weight.

7. A mycobactericidal composition as defined in claim 1 wherein the monohydric alcohol is selected from C₂-C₃ monohydric alcohols and combinations thereof.

8. A mycobactericidal composition as defined in claim 1 wherein the benzoic acid is present in the composition at a concentration between about 0.01% and about 20% by weight.

9. A mycobactericidal composition as defined in claim 8 wherein the benzoic acid is present in the composition at a concentration between about 0.03% and about 5% by weight.

10. A mycobactericidal composition as defined in claim 8 wherein the benzoic acid is present in the composition at a concentration less than 1% by weight.

11. A mycobactericidal composition as defined in claim 1 further comprising surfactant.

12. A mycobactericidal composition as defined in claim 11, wherein the surfactant content is less than about 1 wt %.

13. A mycobactericidal composition as defined in claim 11 wherein the surfactant content is less than about 0.25 wt %.

14. A mycobactericidal composition as defined in claim 11 wherein the surfactant is selected from the group consisting of nonionic surfactants, anionic surfactants and combinations of two or more of the foregoing.

15. A mycobactericidal composition as defined in claim 14 wherein the nonionic surfactant is selected from the group consisting of alcohol ethoxylates, betaines, glucosides, fatty acid esters, amine oxides, sorbitan esters, block copolymers of ethylene oxide and propylene oxide, and combinations of two or more of the foreogoing.

16. A mycobactericidal composition as defined in claim 14 wherein the anionic surfactant is selected from the group consisting of alpha olefin sulfonates, alkyl benzene sulfonates, alkyl sulfates, fatty alcohol ethoxylate sulfonates, ester sulfosuccinates, diesters of sulfosuccinic esters, salts of fatty acids and combinations of two or more of the foregoing.

17. A mycobactericidal composition as defined in claim 14 wherein the anionic surfactant is sodium dioctyl sulfosuccinate.

18. A mycobactericidal composition as defined in claim 14, wherein the surfactant is anionic, the composition further comprising one or more counter-irritant, the counter-irritant comprising block copolymer of ethylene oxide and propylene oxide.

20. A mycobactericidal composition as defined in claim 1, further comprising sporicidal agent.

21. A mycobactericidal composition as defined in claim 20, wherein sporicidal agent is selected from the group consisting of hydrogen peroxide, peracids, peresters, chlorine, iodine, povidone iodine, aldehydes and combinations of two or more of the foregoing.

22. A mycobactericidal composition as defined in claim 20, wherein sporicidal agent is present in the composition at a concentration from 0 to about 10% by weight.

23. A mycobactericidal composition as defined in claim 20, wherein sporicidal agent is present in the composition at a concentration from 0 to about 3% by weight.

24. A mycobactericidal composition as defined in claim 20, wherein sporicidal agent is hydrogen peroxide at a concentration within the range from about 2% to about 8% by weight.

25. A mycobactericidal composition as defined in claim 1 having a pH ranging from about 3.5 to about 6.5.

26. A method for disinfecting a surface, comprising the steps of:
Applying the composition of claim 1 to a surface.

27. The method defined in claim 26, further comprising removing the composition from the surface.

28. The method as defined in claim 26 wherein the surface is a lumen of a medical device.

29. The method as defined in claim 28 wherein the medical device is an endoscope.

30. The method as defined in claim 26 wherein, following the applying step, the composition is allowed to remain in contact with the surface for at least about 5 minutes; and removing the composition from the surface.

31. The method as defined in claim 26 wherein, following the applying step, the composition is allowed to remain in contact with the surface for less than about 5 minutes; and removing the composition from the surface.

32. A method for inactivating mycobacteria, bacteria, virus or fungi using the composition of claim 1.

33. A method for reconditioning a soiled endoscope, comprising:
a first cleaning step to clean the surfaces of the endoscope;
leak testing the endoscope;
a second cleaning step to further clean the surfaces of the endoscope;
disinfecting the surfaces of the instrument by applying the composition of claim 1 to the surfaces for a period of time;
rinsing the surfaces of the endoscope with water; and
drying the endoscope.

34. The method as defined in claim 33 wherein the disinfecting step is performed while the surfaces of the endoscope are at ambient temperature, the composition being applied to the surfaces for about 8 minutes or less.

35. The method as defined in claim 33 wherein the disinfecting step is performed while the surfaces of the endoscope are at an elevated temperature.
